(19) United States
(12) Patent Application Publication
(10) Pub. No.: US 2020/0121818 A1
(43) Pub. Date: Apr. 23, 2020

(54) Sterilization Gas Cleaning Closed System Device

(71) Applicant: Hitachi Industrial Equipment Systems Co., Ltd., Chiyoda-ku, Tokyo (JP)

(72) Inventor: Takeshi KANEKO, Tainai (JP)

(21) Appl. No.: 16/627,910

(22) PCT Filed: Feb. 4, 2019
(86) PCT No.: PCT/JP2019/003899

§ 371 Date: Dec. 31, 2019

(30) Foreign Application Priority Data
Apr. 24, 2018 (JP) 2018-083427

(51) Int. Cl. A61L 2/20; A61L 2/26

(52) U.S. Cl. CPC A61L 2/208; A61L 2/26; A61L 2202/122; A61L 2202/123; A61L 2202/13; A61L 2202/14; A61L 2202/121

(57) ABSTRACT

An object of the present invention is to provide a sterilization gas purifying closed system device that is capable of performing a sterilization process with an inexpensive configuration. In order to achieve the object, there is provided a sterilization gas purifying closed system device that performs a sterilization process on a sealed use area using a sterilization gas, the device including: a gas pipe connected to the use area via a first opening that is open or closed by opening or closing a first valve; and a catalyst area connected to the use area via a second opening that is open or closed by opening or closing a second valve, in which the sterilization gas is a hydrogen peroxide gas, in which platinum and palladium are provided as a catalyst in the catalyst area, in which when the sterilization process is performed, if the second valve is closed and the first valve is open, the hydrogen peroxide gas is introduced into the use area from the gas pipe via the first opening, and after the introduction, if the first valve is closed and the use area is brought into a sealed state, the sterilization process is performed using the hydrogen peroxide gas, and in which after a predetermined period of time, if the first valve and the second valve are open and a non-toxic gas is introduced from the gas pipe via the first opening, the hydrogen peroxide gas in the use area is introduced, in a push manner, into the catalyst area, and the hydrogen peroxide gas is purified.

### TECHNICAL FIELD

The present invention relates to a closed system device such as a biosafety cabinet that eliminates, using an air barrier, a contamination risk from outside, or a pass box which prevents the entry of outside contaminated air and through which an article or equipment is taken in and out.

### BACKGROUND ART

In the related art, in the industrial fields such as the medical field and the pharmaceutical field, a biosafety cabinet is used as a countermeasure against biological hazard. The biosafety cabinet has the isolation capability of being able to protect a specimen from sundry bacteria from outside by providing an air barrier and performing an operation in a partitioned space having an opening portion in a portion thereof.

Patent Document 1 and Patent Document 2 disclose the background art of the technical field. Patent Document 1 discloses a biosafety cabinet that includes a coupling portion connected to an exhaust port of the biosafety cabinet, an opening portion which is different from the coupling portion and into which air flows, and an open type duct having an exhaust duct, the cabinet includes a differential pressure sensor that detects a difference between a pressure in a space where the open type duct is disposed and a pressure in the open type duct, and detection means for outputting a detection signal when an absolute value of the differential pressure sensor becomes less than or equal to a predetermined threshold value. In addition, Patent Document 2 discloses a point that see-through windows are provided in a portion of a back wall or a side wall of an operation space of a biosafety cabinet and in a portion of a main body rear wall or a main body side wall of the biosafety cabinet which is separated from the back wall or the side wall by a circulation flow path, so as to enable an operator to see through both walls, a display device is installed in an outside part of the see-through window, and the operator inserts the arms from a front opening portion of the biosafety cabinet to perform an operation from a front shutter while seeing the operation space.

### CITATION LIST

### Patent Document

Patent Document 1: JP 2017-078527 A
Patent Document 2: JP 2016-165249 A

### SUMMARY OF THE INVENTION

### Problems to be Solved by the Invention

As shown in Patent Document 1 and Patent Document 2, in a closed system device such as the biosafety cabinet for safely performing an operation while maintaining an inside purity, or the pass box which prevents the entry of outside contaminated air and through which an article or equipment is taken in and out, a sterilization process using a sterilization gas such as hydrogen peroxide gas is effective in sterilizing cultured cells, adhering excess contaminants, and the like. However, since the hydrogen peroxide gas is toxic, when the hydrogen peroxide gas is exhausted, the hydrogen peroxide gas needs to be guided outside a building through a duct and to be discharged outside, and thus a pipe installation costs too much, which is a problem.

The present invention is made in light of the problem, and an object of the present invention is to provide a sterilization gas purifying closed system device that is capable of performing a sterilization process with an inexpensive configuration.

### Solutions to Problems

The present invention is made in light of the background art and the problem, and as an example of the present invention, there is provided a sterilization gas purifying closed system device that performs a sterilization process on a sealed use area using a sterilization gas, the device including: a gas pipe connected to the use area via a first opening that is open or closed by opening or closing a first valve; and a catalyst area connected to the use area via a second opening that is open or closed by opening or closing a second valve, in which the sterilization gas is a hydrogen peroxide gas, in which platinum and palladium are provided as a catalyst in the catalyst area, in which when the sterilization process is performed, if the second valve is closed and the first valve is open, the hydrogen peroxide gas is introduced into the use area from the gas pipe via the first opening, and after the introduction, if the first valve is closed and the use area is brought into a sealed state, the sterilization process is performed using the hydrogen peroxide gas, and in which after a predetermined period of time, if the first valve and the second valve are open and a non-toxic gas is introduced from the gas pipe via the first opening, the hydrogen peroxide gas in the use area is introduced, in a push manner, into the catalyst area, and the hydrogen peroxide gas is purified.

### Effects of the Invention

According to the present invention, it is possible to provide a sterilization gas purifying closed system device that is capable of performing a sterilization process with an inexpensive configuration.

### BRIEF DESCRIPTION OF THE DRAWINGS

FIG. 1 is a schematic front view of a biosafety cabinet in the related art.

FIG. 2 is a schematic side cross-sectional view of the biosafety cabinet when a cross section A-A′ in FIG. 1 is seen from right.

FIG. 3 is a view illustrating an air flow when the biosafety cabinet in FIG. 2 operates.

FIG. 4 is a schematic front view of an example of a clean air system in the related art.

FIG. 5 is a schematic cross-sectional view of a sterilization gas purifying closed system device in Example 1.

FIG. 6 is a schematic cross-sectional view of a catalyst layer in Example 1.

FIG. 7 is a schematic cross-sectional view of a sterilization gas purifying closed system device in Example 2.

### MODE FOR CARRYING OUT THE INVENTION

Hereinafter, examples of the present invention will be described with reference to the drawings.

### Example 1

First of all, initially, a biosafety cabinet in the related art which is the premise of the present example will be described.

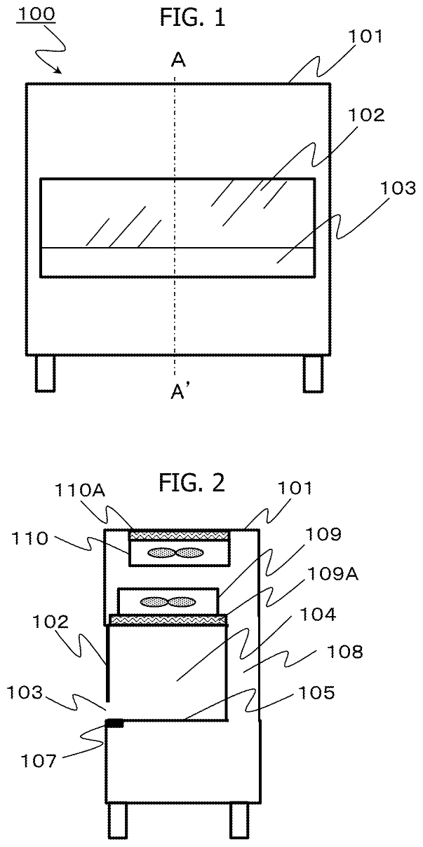

FIG. 1 illustrates a schematic front view of the biosafety cabinet. In addition, FIG. 2 illustrates a schematic side cross-sectional view of the biosafety cabinet when a cross section A-A′ in FIG. 1 is seen from right.

An opening is provided in a central area of a housing 101 of a biosafety cabinet 100, and an operation space 104 is provided therebehind. A front plate 102 is provided on a front surface side of the operation space 104 so as to block an upper portion of the opening, an operation opening 103 is provided on a lower side thereof, and an operator inserts the hands into the operation space 104 from the operation opening 103 to perform an operation. The front plate 102 is formed of a transparent material such as glass, and the operator can see an operation through the front plate.

An operation stage 105 which is substantially planar is provided on a bottom surface of the operation space 104, and the operator performs an operation on the operation stage. An air intake port 107 leading downward is provided close to the operation opening 103 on a front side of the operation stage 105. The air intake port 107 is formed of, for example, a slit that extends along the operation opening 103 in a rightward and leftward direction of the housing. A back flow path 108 leading from the air intake port 107 to an upper portion of the housing is provided on a back surface side of the operation space 104.

An air blow side fan filter unit (FFU) 109 is provided above the operation space 104. The air blow side FFU 109 is formed of a fan which is rotated by a motor, and a filter, for example, a HEPA filter 109A which removes microparticles, and blows purified air, from which the microparticles are removed, into the operation space 104. An exhaust side fan filter unit (FFU) 110 is provided in the upper portion of the housing 101, and removes microparticles from a portion of air with a filter, for example, a HEPA filter 110A to exhaust the portion of air outside the device.

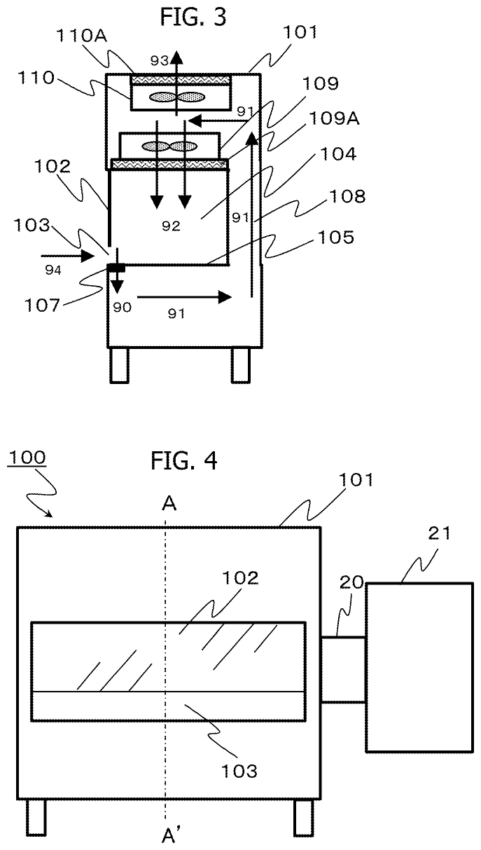

In FIG. 3, an air flow when the biosafety cabinet operates is illustrated with the arrows. An air flow 90 which is taken in from the air intake port 107 on a front surface side of the operation stage 105 is blown, as shown with reference sign 91, into the operation space 104 from the air blow side FFU 109 through a lower portion of the housing, the back flow path 108, and the upper portion of the housing. Since purified air from which microparticles are removed by the HEPA filter 109A of the air blow side FFU 109 is blown into the operation space 104, the operation space 104 is maintained in a purified state. At the time, if only an air flow denoted by reference sign 92 is blown into the operation space 104, air in the operation space leaks outside, which is a concern. For this reason, the exhaust side FFU 110 is provided, and a portion of air is discharged outside through the HEPA filter 110A. Therefore, a pressure in the operation space decreases, and an air flow 94 to be introduced from outside to inside through the operation opening 103 in a lower part of the front plate 102 is formed. If the air flow 94 flows directly into the operation space, the purity of the operation space decreases. However, since all of the air flow 94 flowing in from the operation opening 103 and the majority of the air flow 92 blown into the operation space are taken in from the air intake port 107 by properly controlling the air volume of the air flow 92 which is blown into the operation space from the air blow side FFU 109, and the air volume of an air flow 93 which is exhausted outside from the exhaust side FFU 110, owing to the air flow 92 blown into the operation space 104, an atmospheric barrier (air barrier) is formed to prevent the air flow 94 from flowing into the operation space 104 from the operation opening 103. Therefore, it is possible to realize an equilibrium where the operation space 104 is not contaminated by air from outside and no air leaks outside before the inside is purified.

Therefore, even though the operator inserts the hands into the operation space 104 through the operation opening 103 to perform an operation, it is possible to realize the maintenance of the purity and the prevention of contamination.

FIG. 4 illustrates a schematic front view of an example of a clean air system. In FIG. 4, the clean air system is a system where the biosafety cabinet 100 is connected to an incubator 21 via a pass box 20. The incubator 21 is a room that is used for culture, incubation, the maintenance of an enzyme reaction, or the like while keeping temperature constant. In addition, the pass box 20 has the function of preventing the entry of outside contaminated air, and enabling the taking in and out of articles or equipment therethrough. As described above, in the clean air system, for example, even though a process such as a cell culture container being handed over between the incubator 21 and the biosafety cabinet 100 is performed, since the entry of outside contaminated air is prevented by the pass box 20, it is possible to reduce a contamination risk. Incidentally, the clean air system may be a device where operation spaces of two biosafety cabinets are coupled to each other via the pass box 20, or may be a system where the inside and the outside of a clean room are connected to each other via the pass box.

In a closed system device such as the biosafety cabinet for safely performing an operation while maintaining an inside purity, the pass box, or the incubator which are described above, a sterilization process using a sterilization gas such as hydrogen peroxide gas is effective in sterilizing cultured cells, adhering excess contaminants, and the like. However, since the hydrogen peroxide gas is toxic, when the hydrogen peroxide gas is exhausted, the hydrogen peroxide gas needs to be guided outside a building through a duct and to be discharged outside, and thus a pipe installation costs too much, which is a problem.

Then, in order to solve the problem, in a configuration of the present example, the hydrogen peroxide gas is detoxified with a catalyst, and thereafter, the detoxified hydrogen peroxide gas is released into a room. Hereinafter, details thereof will be described.

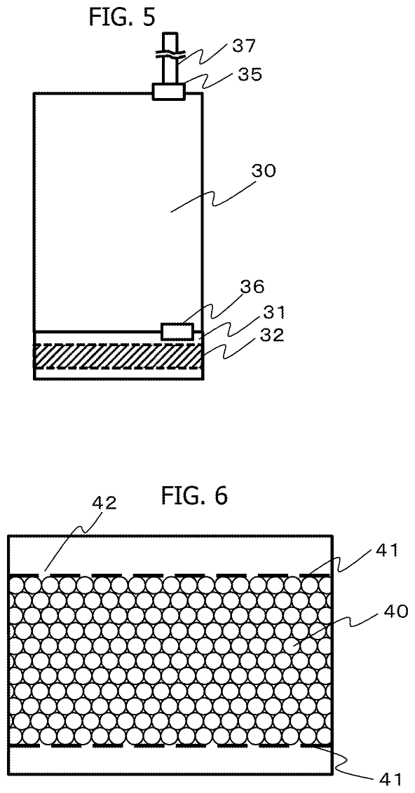

FIG. 5 is a schematic cross-sectional view of a sterilization gas purifying closed system device in the present example. In FIG. 5, 30 is a use area which becomes a closed system when the use area is used, and corresponds to, for example, an area in the pass box 20 described above, an area in the incubator 21 described above, or the operation space 104 which can be a closed system by enclosing and sealing the front plate 102 of the biosafety cabinet 100 described above with rubber or like and providing the interlock mechanism.

31 is a catalyst area, and is an area including a catalyst layer 32 to be described later which detoxifies the hydrogen peroxide gas. The use area 30 has openings 35 and 36. The openings 35 and 36 have a valve 1 and a valve 2, respectively. The openings 35 and 36 are open or closed by opening or closing the valve 1 and the valve 2. The opening 35 and the valve 1, or the opening 36 and the valve 2 may be provided in areas which are apart from each other on a path. In addition, 37 is a gas pipe, and supplies gas from a hydrogen peroxide gas supply source (not illustrated).

When a sterilization process is performed on the use area 30, the valve 1 of the opening 35 is open, and the hydrogen peroxide gas is introduced into the use area 30 via the gas pipe 37. After the introduction, the valve 1 of the opening 35 is closed, and the use area 30 enters a sealed state.

After the sterilization process is performed using the hydrogen peroxide gas for a predetermined period of time, the valve 2 of the opening 36 is open, and also the valve 1 of the opening 35 is open and at the time, a non-toxic gas such as purified air or N2 gas is introduced from the gas pipe 37. Therefore, the hydrogen peroxide gas in the use area 30 is introduced, in a push manner, into the catalyst area 31.

Manganese dioxide, iron oxide, or the like can also be used as the catalyst; however, particularly, platinum and palladium are desirable. The catalyst is granular small single crystal particulates, and has outstanding characteristics such as a high performance to decompose the hydrogen peroxide gas and not generating dust. For use in purified air, it is very important to not generate dust and to not reduce cleanness, and particularly in this point, the catalyst is desirable.

FIG. 6 is a schematic cross-sectional view of the catalyst layer 32 in the present example. In FIG. 6, 41 is a SUS plate having a mesh-shaped opening portion 42, and a catalyst 40 having a particulate shape is held in a stack manner. The hydrogen peroxide gas comes into contact with the catalyst 40 via the mesh-shaped opening portion 42, and a purification process is performed. The hydrogen peroxide gas (H2O2) is converted into H2O and O2, and is detoxified through the purification.

Incidentally, it is desirable that a concentration sensor for the hydrogen peroxide gas is installed in the use area 30, and whether or not the concentration is less than or equal to a threshold value is displayed by a lamp or a display. In addition, it is desirable to adopt a configuration where the use area 30 has a door with the interlock mechanism, the introduction of the hydrogen peroxide gas into the use area 30 is performed only in a sealed state (door closed state), and until the concentration of the hydrogen peroxide gas becomes less than or equal to a reference value, the lock of the door is not released owing to the interlock, namely, the hydrogen peroxide gas is not released to atmosphere.

As described above, since the present example is configured such that the hydrogen peroxide gas is detoxified with the catalyst, and thereafter, the detoxified hydrogen peroxide gas is released to the room, it is possible to provide the sterilization gas purifying closed system device that is capable of performing a sterilization process with an inexpensive configuration.

### Example 2

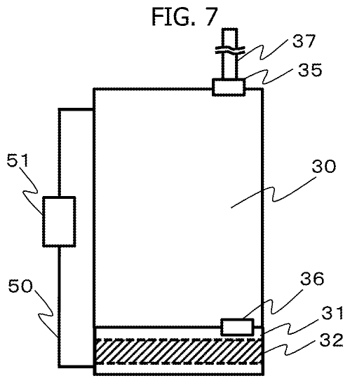

FIG. 7 is a schematic cross-sectional view of a sterilization gas purifying closed system device in the present example. In FIG. 7, the same reference signs are assigned to configuration elements having the same functions as those of the configuration elements in FIG. 5, and descriptions thereof will be omitted. In FIG. 7, the point of difference from FIG. 5 is that the catalyst area 31 is connected to the use area 30 via a circulation pipe 50, and gas in the circulation pipe 50 is circulated by a circulation fan 51.

Namely, as illustrated in FIG. 7, a gas passing through the catalyst 40 in the catalyst area 31 is introduced to the use area 30 through the circulation pipe 50, and a gas or the hydrogen peroxide gas in the use area 30 is pushed again toward the catalyst area 31 by the gas in the circulation pipe 50. Therefore, over time, the total amount of the hydrogen peroxide gas coming into contact with the catalyst 40 increases, and the number of contacts with the catalyst increases, and thus the effect of the purification process improves.

Incidentally, it is desirable that an inlet of the circulation pipe 50 for a gas passing through the catalyst 40 is disposed on a side in the catalyst area 31 which the hydrogen peroxide gas passing through the catalyst layer 32 reaches, namely, below the catalyst layer 32 of the catalyst area 31 illustrated in FIG. 7, and an outlet of the circulation pipe 50 for the gas passing through the catalyst is disposed on a side in the use area 30 into which the hydrogen peroxide gas is introduced from the opening 35, namely, on an upper side of the use area 30 illustrated in FIG. 7.

According to the present example, it is possible to improve the effect of the purification process by circulating the gas, which passes through the catalyst 40, between the use area 30 and the catalyst area 31 through the circulation pipe 50.

The examples have been described above; however, the present invention is not limited to the examples, and includes various modification examples. For example, the examples have been described in detail so as to facilitate the understanding of the present invention, and the present invention is not necessarily limited to including all of the described configurations. In addition, a portion of a configuration of an example can be replaced with a configuration of another example. In addition, a configuration of another example can also be added to a configuration of an example. In addition, the addition, the deletion, or the replacement of another configuration can be made with respect to a portion of a configuration of each example.

### REFERENCE SIGNS LIST

20 Pass box
21 Incubator
30 Use area
31 Catalyst area
32 Catalyst layer
35, 36 Opening
37 Gas pipe
40 Catalyst
41 SUS plate
42 Opening portion
50 Circulation pipe
51 Circulation fan
100 Biosafety cabinet
101 Housing
102 Front plate
103 Operation opening
104 Operation space
105, 105T Operation stage
107 Air intake port
108 Back flow path
109 Air blow side fan filter unit (FFU)
109A Air blow side HEPA filter
110 Exhaust side fan filter unit (FFU)
110A Exhaust side HEPA filter

1. A sterilization gas purifying closed system device that performs a sterilization process on a sealed use area using a sterilization gas, the device comprising:
a gas pipe connected to the use area via a first opening that is open or closed by opening or closing a first valve; and
a catalyst area connected to the use area via a second opening that is open or closed by opening or closing a second valve,
wherein the sterilization gas is a hydrogen peroxide gas,
wherein platinum and palladium are provided as a catalyst in the catalyst area,
wherein when the sterilization process is performed on the use area, if the second valve is closed and the first valve is open, the hydrogen peroxide gas is introduced into the use area from the gas pipe via the first opening, and after the introduction, if the first valve is closed and the use area is brought into a sealed state, the sterilization process is performed using the hydrogen peroxide gas, and
wherein after a predetermined period of time, if the first valve and the second valve are open and a non-toxic gas is introduced from the gas pipe via the first opening, the hydrogen peroxide gas in the use area is introduced, in a push manner, into the catalyst area, and the hydrogen peroxide gas is purified.

2. The sterilization gas purifying closed system device according to claim 1,
wherein in the catalyst, the platinum and the palladium which have a particulate shape are held with a SUS plate having a mesh-shaped opening portion.

3. The sterilization gas purifying closed system device according to claim 1, further comprising:
a circulation pipe that connects the catalyst area to the use area; and
a circulation fan that circulates a gas in the circulation pipe,
wherein a gas passing through the catalyst in the catalyst area is introduced to the use area through the circulation pipe, and a gas in the use area is pushed toward the catalyst area by the gas in the circulation pipe.

4. The sterilization gas purifying closed system device according to claim 3,
wherein an inlet of the circulation pipe for the gas passing through the catalyst is disposed on a side in the catalyst area which the hydrogen peroxide gas passing through the catalyst reaches, and an outlet of the circulation pipe for the gas passing through the catalyst is disposed on a side in the use area into which the hydrogen peroxide gas is introduced from the first opening.

5. The sterilization gas purifying closed system device according to claim 1,
wherein a concentration sensor for the hydrogen peroxide gas is installed in the use area, and
wherein whether or not a numerical value of the concentration sensor is less than or equal to a threshold value is displayed by a lamp or a display.

6. The sterilization gas purifying closed system device according to claim 5,
wherein the use area has a door with an interlock mechanism, and
wherein owing to the interlock mechanism, an introduction of the hydrogen peroxide gas into the use area is performed only in a sealed state where the door is closed, and until a concentration of the hydrogen peroxide gas becomes less than or equal to a reference value, the door is locked.

7. The sterilization gas purifying closed system device according to claim 1,
wherein the use area is a pass box.

8. The sterilization gas purifying closed system device according to claim 1,
wherein the use area is an incubator.

9. The sterilization gas purifying closed system device according to claim 1,
wherein the use area is an operation space that can be a closed system by enclosing and sealing a front plate of a biosafety cabinet and providing the interlock mechanism.
